SEQUENCE LISTING (32 sequences)

SEQ ID NO: 1 - DNA, 20 bp, artificial sequence
  the nucleotide sequence of gRNA/gRNA-miR-29b-D
cctaaaacactgatttcaaa

SEQ ID NO: 2 - RNA, 23 nt, artificial sequence
  the nucleotide sequence of the miR-29b
uagcaccauuugaaaucaguguu

SEQ ID NO: 3 - RNA, 57 nt, artificial sequence
  the nucleotide sequence of pre-miR-29b
aggaagcugguuucauauggugguuuagauuaaauaguagcaccauuugaaaugucu

SEQ ID NO: 4 - DNA, 20 bp, artificial sequence
  gRNA-miR-29b-A
aggaagctggtttcatatgg

SEQ ID NO: 5 - DNA, 20 bp, artificial sequence
  gRNA-miR-29b-B
ttcaggaagctggtttcata

SEQ ID NO: 6 - DNA, 20 bp, artificial sequence
  gRNA-miR-29b-C
ccatttgaaatcagtgtttt

SEQ ID NO: 7 - DNA, 19 bp, artificial sequence
  miR-29b-1 F
gctgcaccgtgaatggtaa

SEQ ID NO: 8 - DNA, 20 bp, artificial sequence
  miR-29b-1 R
aggtcttcatccgagcatgg

SEQ ID NO: 9 - DNA, 25 bp, artificial sequence
  miR-29b-2 F
tgtacatatgttgaatggatttggt

SEQ ID NO: 10 - DNA, 20 bp, artificial sequence
  miR-29b-2 R
tgctgcaaccaggactgaat

SEQ ID NO: 11 - DNA, 20 bp, artificial sequence
  Upstream primer F
tacaacgccctgaatgacct

SEQ ID NO: 12 - DNA, 19 bp, artificial sequence
  A downstream primer R
gtcctcttcgttgaccgga

SEQ ID NO: 13 - DNA, 21 bp, artificial sequence
  chr1+196863288 Forward primer
gggactcgagcactctatgtt

SEQ ID NO: 14 - DNA, 20 bp, artificial sequence
  chr1+196863288 Reverse primer
agggtcattagcagggttcc

SEQ ID NO: 15 - DNA, 20 bp, artificial sequence
  chr9-101867160 Forward primer
tcatcggaccttgacagctc

SEQ ID NO: 16 - DNA, 20 bp, artificial sequence
  chr9-101867160 Reverse primer
tgtgccaggccagagaaaaa

SEQ ID NO: 17 - DNA, 19 bp, artificial sequence
  chr2-44912669 Forward primer
agccgagctatcaatgggc

SEQ ID NO: 18 - DNA, 21 bp, artificial sequence
  chr2-44912669 Reverse primer
ttcatctgcattctgcgctgt

SEQ ID NO: 19 - DNA, 20 bp, artificial sequence
  chr11-26661876 Forward primer
ctgcattgagtgccttagcg

SEQ ID NO: 20 - DNA, 21 bp, artificial sequence
  chr11-26661876 Reverse primer
gaccatttggaaaccgtgtga

SEQ ID NO: 21 - DNA, 20 bp, artificial sequence
  chr5-68275574 Forward primer
gcactggggacataggtgag

SEQ ID NO: 22 - DNA, 20 bp, artificial sequence
  chr5-68275574 Reverse primer
agcccaccttggcaatagac

SEQ ID NO: 23 - DNA, 21 bp, artificial sequence
  chrX+93743993 Forward primer
cagtgagcttcacagtttgct

SEQ ID NO: 24 - DNA, 24 bp, artificial sequence
  chrX+93743993  Reverse primer
gagtcatacagtatttaggctgct

SEQ ID NO: 25 - DNA, 20 bp, artificial sequence
  chr16+15872481 Forward primer
aaggctgaatgccgttcact

SEQ ID NO: 26 - DNA, 20 bp, artificial sequence
  chr16+15872481 Reverse primer
ggcaagaagaacctgggaca

SEQ ID NO: 27 - DNA, 20 bp, artificial sequence
  chr15-66654630 Forward primer
atgcagcagatgccagactt

SEQ ID NO: 28 - DNA, 20 bp, artificial sequence
  chr15-66654630 Reverse primer
ctcatgagcacaggagccaa

SEQ ID NO: 29 - DNA, 20 bp, artificial sequence
  chr11-98951837 Forward primer
gaccaccacaatcggctgta

SEQ ID NO: 30 - DNA, 20 bp, artificial sequence
  chr11-98951837  Reverse primer
gttcttggctcccctgactc

SEQ ID NO: 31 - DNA, 20 bp, artificial sequence
  chr4+40629962 Forward primer
tggctgccaatacctatgct

SEQ ID NO: 32 - DNA, 20 bp, artificial sequence
  chr4+40629962 Reverse primer
agccatcccctcgactcaaa

(19) United States
(12) Patent Application Publication
(10) Pub. No.: US 2022/0127602 A1
(43) Pub. Date: Apr. 28, 2022

(54) GRNA TARGETING MIR-29B, AAV8-CRISPER/CAS9 SYSTEM AND USE THEREOF

(71) Applicant: Shanghai University, Shanghai (CN)

(72) Inventors: Junjie XIAO, Shanghai (CN); Jin LI, Shanghai (CN); Yihua BEI, Shanghai (CN)

(21) Appl. No.: 17/077,762

(22) Filed: Oct. 22, 2020

(51) Int. Cl. C12N 15/11; C12N 9/22; C12N 15/86

(52) U.S. Cl. CPC C12N 15/11; C12N 9/22; C12N 2830/001; C12N 2310/20; C12N 2750/14143; C12N 15/86

(57) ABSTRACT

The application provides a gRNA targeting miR-29b, an AAV8-CRISPR/Cas9 system and use thereof, belonging to the technical field of genetic engineering. The application provides a gRNA targeting miR-29b, which has a nucleotide sequence set forth in SEQ ID No.1. The gRNA shows strong specific editing ability to miR-29b at the cellular level, only has targeted editing effect on miR-29b without influence on miR-29a and miR-29c of the same family, and also shows higher editing efficiency, thus effectively inhibiting the expression of miR-29b. An AAV8-CRISPR/Cas9 system containing said gRNA targeting miR-29b. The use of gRNA targeting miR-29b or AAV8-CRISPR/Cas9 system in preparing drugs for treating muscle atrophy.

### TECHNICAL FIELD

The application belongs to the technical field of genetic engineering, especially relating to a gRNA targeting miR-29b, an AAV8-CRISPER/Cas9 system and use thereof.

### BACKGROUND ART

Skeletal muscle is the largest organ of human body, which, as a target organ dominated by nervous system, plays an important role in the process of human movement. Muscle atrophy is a progressive disease of skeletal muscle cell degeneration, and the main manifestation of muscle atrophy is a decrease of muscle mass. Patients with limited mobility or disability are often accompanied by muscle atrophy, such as septicemia, cancer, AIDS, congestive heart failure, chronic obstructive pulmonary disease, renal failure, severe burns and Cushing's Disease can cause muscle atrophy. At present, exercise rehabilitation is still the main treatment for muscle atrophy in clinical practice. Unfortunately, exercise rehabilitation is not suitable for patients with paralysis, disability, end-stage heart failure and severe burns, while as for patients with cancer and renal failure, even increasing exercise may not completely reverse the occurrence of muscle atrophy.

Adeno-associated virus (AAV) belongs to the Parvoviridae dependent virus genus. At present, more than 40 different serotypes have been identified, the serotypes of AAV are distinguished by the differences of their protein capsids. Currently, the known AAV types include AAV1-9, avian AAV, Bovine AAV, canine AAV etc. AAV, as a vector of gene therapy, has many advantages, such as low pathogenicity, being able to invade and infect cells in division stage and quiescent stage, also being able to stably exist in host cells and express target genes for a long time.

CRISPR/Cas9 system was first found in bacteria, when bacteria are infected by phage, CRISPR/Cas9 system may effectively recognize and cut foreign DNA, based on its recognition and cutting effect on target DNA. CRISPR/Cas9 provides a simple and efficient method for genetic editing, which is quickly developed for the treatment of hereditary diseases, cancers, viral infectious diseases and many diseases related to gene mutation.

miRNA is a short non-coding endogenous RNA, usually with 18˜25 nucleotides, which regulates the expression of target genes by binding to the 3′UTR of mRNA and inhibiting its translation. The precursors of miRNA are firstly transcribed in the nucleus, and then matured through several enzymatic reactions and transferred to cytoplasm to play their biological functions. miRNA plays its regulatory role by recruiting specific silencing proteins to form RNA-induced silencing complex (RISC). It is speculated that in humans, about 60% of mRNA is the target of miRNA, and one miRNA may target more than 100 mRNAs. Previous studies have shown that non-coding RNA plays an important role in the occurrence and development of muscle atrophy. Studies have shown that various miRNAs such as miR-1, miR-29b, miR-133, miR-23a, miR-21, miR-27, miR-628, miR-431, miR-206 and the like play a regulatory role in the occurrence of muscle atrophy. However, there is no long-acting and safe gene therapy for muscle atrophy.

### SUMMARY OF THE APPLICATION

Therefore, the purpose of the application is to provide a gRNA targeting miR-29b, an AAV8-CRISPER/Cas9 system and use thereof, the gRNA may effectively inhibit the expression of miR-29b.

The purpose of the application is to provide an AAV8-CRISPR/Cas9 system targeting miR-29b and use thereof. The system may target miR-29b in a long-acting and safe way in skeletal muscle cells, and realize the purpose of preparing drugs for gene therapy of muscle atrophy by inhibiting the expression of miR-29b.

The application provides a gRNA targeting miR-29b, the nucleotide sequence of the gRNA is set forth in SEQ ID No.1.

In an embodiment, the nucleotide sequence of the miR-29b is set forth in SEQ ID No.2.

The application provides an AAV8-CRISPR/Cas9 system targeting miR-29b, including said gRNA.

In an embodiment, the gRNA is expressed under dMCK promoter in the AAV8-CRISPR/Cas9 system.

The application provides a method for constructing the AAV8-CRISPR/Cas9 system, including the following steps:

using restriction enzyme to cut an adeno-associated virus plasmid, obtaining a linear adeno-associated virus plasmid;

inserting the gRNA into the linear adeno-associated virus plasmid, obtaining the AAV8-CRISPR/Cas9 system.

In an embodiment, the adeno-associated virus plasmid is pAAV-dMCK-SACas9-PA-gRNA.

In an embodiment, the restriction enzyme is BbsI.

The application provides use of gRNA, AAV8-CRISPR/Cas9 system, or AAV8-CRISPR/Cas9 system constructed by said method in preparing drugs for treating muscle atrophy.

In an embodiment, the muscle atrophy includes myogenic muscle atrophy, disused muscle atrophy or muscle atrophy induced by chronic disease heart failure.

The application provides a gRNA targeting miR-29b, the nucleotide sequence of the gRNA is set forth in SEQ ID No.1. Compared with other designed gRNAs, the gRNA provided by the application shows strong specific binding ability to miR-29b at the cellular level, and shows higher editing efficiency, which may effectively inhibit the expression of miR-29b, and only has a targeted editing effect on miR-29b with no influence on miR-29a and miR-29c of the same family. The T7 enzymatic digestion verification test result shows that the gRNA provided by the application has no off-target effect.

The application provides an AAV8-CRISPR/Cas9 system targeting miR-29b, which includes the gRNA. Taking miR-29b, which is highly expressed in various kinds of muscle atrophy, as the target, CRISPR-Cas9 (initiated by muscle-specific promoter dMCK) is delivered to skeletal muscle cells through AAV8 virus vector to proceed gene editing for miR-29b in skeletal muscle cells, and specifically reduces the expression of miR-29b in skeletal muscle, thus realizing the treatment of muscle atrophy. The mouse grip strength test shows that AngII induces muscle atrophy in gastrocnemius of mice and the grip strength of mice decreases, while the AAV8-CRISPR-Cas9-29b-D virus could effectively reverse muscle atrophy and the grip strength is partially restored. As for the effect of AngII on the myotube diameter, AngII induces muscle atrophy in gastrocnemius of mice and the diameter of gastrocnemius myotube reduces significantly, while AAV8-CRISPR-Cas9-29b-D virus may effectively reverse muscle atrophy and restore the myotube diameter.

Further, the application specifically defines that the gRNA is expressed under dMCK promoter in AAV8-CRISPR/Cas9 system, which ensures that the system only plays a role in skeletal muscle cells, and has no influence on other types of cells and tissues, also has good targeting characteristics and safety.

### BRIEF DESCRIPTION OF THE DRAWINGS

FIGS. 1a-1e are diagrams of screening the optimum gRNA through cellular level experiment, wherein FIG. 1a is a schematic diagram of designing editing sites for miR-29b; FIG. 1b shows the effect of different gRNA on miR-29b expression; FIG. 1c shows T7 digestion to verify the editing efficiency of gRNA-miR-29b-D and gRNA-29b-C; FIG. 1d shows the verification of the specificity of gRNA-miR-29b-D to miR-29b at the cellular level; FIG. 1e is a diagram that verify whether gRNA-miR-29b-D has off-target effect; *, p<0.05, **, p<0.01;

FIGS. 2a-2e show the therapeutical effect of CRISPR/Cas9 system delivered by adeno-associated virus AAV8 on Ang II-induced muscle atrophy. FIG. 2a shows the schematic diagram of AAV8 virus carrying target genes; FIG. 2b shows that the CRISPR-Cas9 system delivered by AAV8 may restore the hind limb grip of Ang II-induced muscle atrophy model mice; FIG. 2c shows that the CRISPR-Cas9 system delivered by AAV8 may inhibit the expression of miR-29b in gastrocnemius muscle of Ang II-induced muscle atrophy model mice; FIG. 2d shows the effect of AAV8-CRISPR-miR-29b-D on gastrocnemius muscle weight in Ang II-induced muscle atrophy model; FIG. 2e shows the effect on myotube diameter after treated with AAV8-CRISPR-miR-29b-D in Ang II-induced muscle atrophy model; *, p<0.05, **, p<0.01;

FIG. 3 is a figure showing a structure of AAV8 vector plasmid pAAV-dMCK-SACas9-PA-gRNA;

FIG. 4 is a standard curve of the virus.

### DETAILED DESCRIPTION OF THE EMBODIMENTS

The application provides a gRNA targeting miR-29b, the nucleotide sequence of the gRNA is set forth in SEQ No.1 (CCTAAAACACTGATTTCAAA).

In an embodiment, the nucleotide sequence of the miR-29b is set forth in SEQ ID No.2 (UAGCACCAUUUGAAAUCAGUGUU). The gene editing for miR-29b is selected from the sequence of pre-miR-29b, and its nucleotide sequence is set forth in SEQ ID No.3: (AGGAAGCUGGUUUCAUAUGGUGGUUUAGAUUAAAUAGUAGCACCAUUUGAAAU GUCU). In the application, there is no special limit on the design method of gRNA, and a design scheme well known in the art will do. In the application, there is no special limit on the source of gRNA targeting miR-29b, and a synthetic method of gRNA well known in the art will do. Compared with other designed gRNAs, the gRNA provided in the application shows strong specific binding ability to miR-29b at the cellular level, it also has higher editing efficiency, which may effectively inhibit the expression of miR-29b. What's more, this gRNA has the specific targeted editing effect on miR-29b, which has no impact on miR-29a and miR-29c of the same family.

The application provides an AAV8-CRISPER/Cas9 system targeting miR-29b, including the gRNA.

In an embodiment, the gRNA is expressed under the dMCK promoter in the AAV8-CRISPER/Cas9 system. Compared with the lack of selectivity of the universal U6 promoter used in lentivirus system, which may also enter other types of cells and cause side effects, dMCK promoter ensures that this system only plays a role in skeletal muscle cells, and has no influence on other types of cells and tissues, also with excellent targeting characteristics.

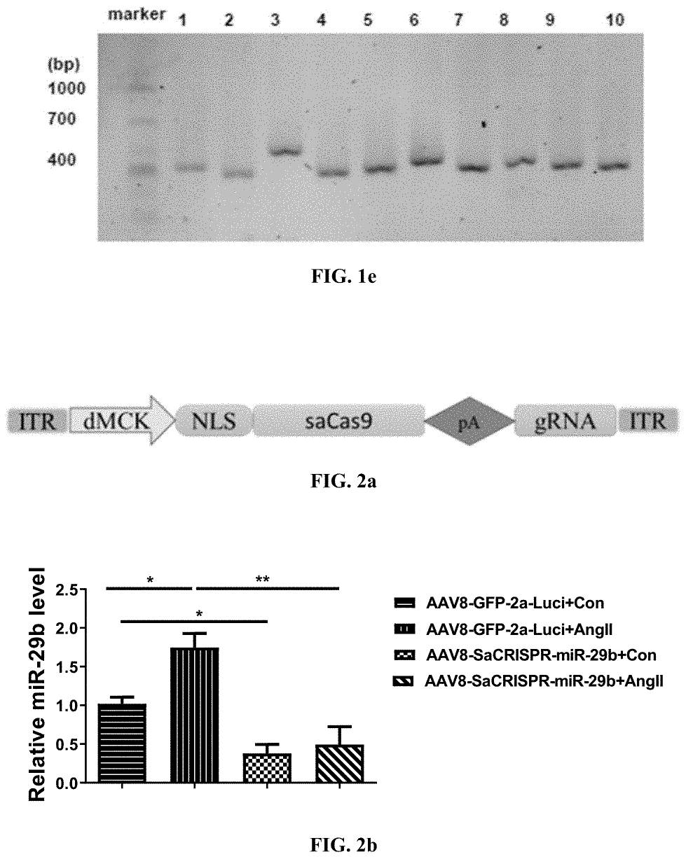

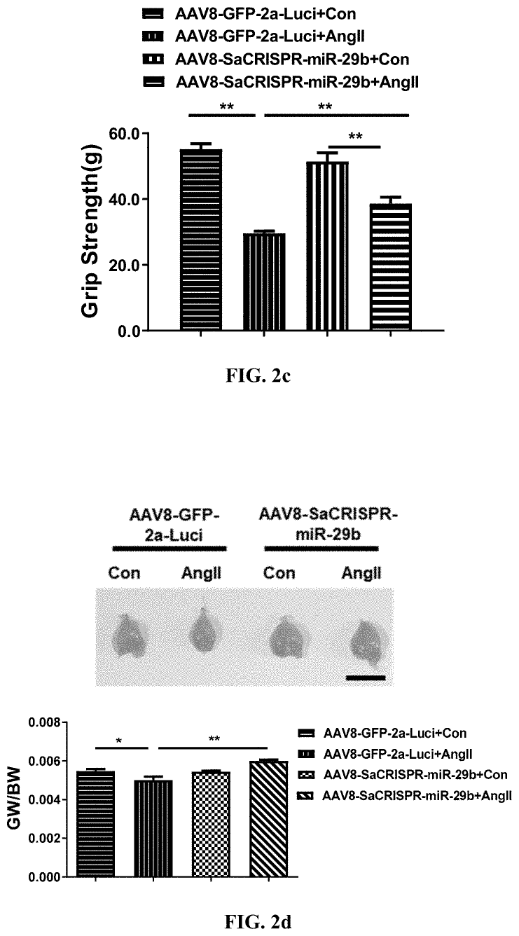

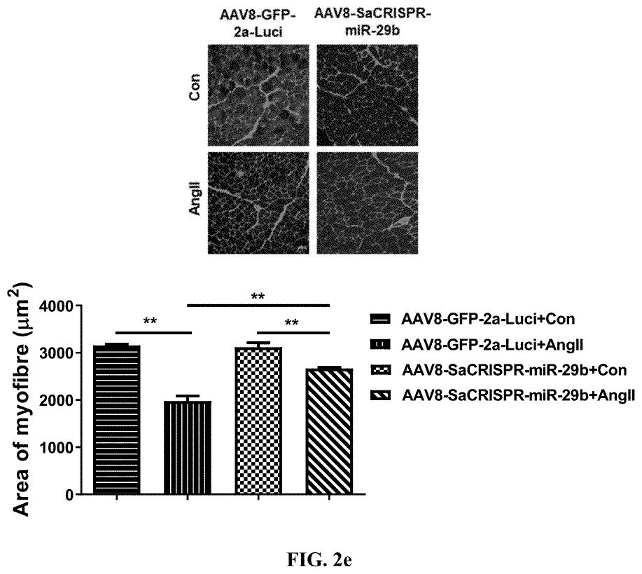

In the application, the schematic diagram of the target gene carried by AAV8 virus is shown in FIG. 2a.

In the application, the lentivirus mediated CRISPR-Cas9-gRNA gene fragment that enters into the host cell is taken as a control, the result shows that its inhibitory effect to miR-29b is relatively fine as well, but because of the mechanism of lentivirus that the gene fragment will integrate into the genome of the host cell, it (the control) is not safe to be used as gene therapy, which is only suitable for the experimental research. Furthermore, the lentivirus has a short-acting time. While the AAV8 has the characteristics that dissociating in cytoplasm rather than integrated, long preservation time and being removable, so it is more suitable for gene therapy.

The application provides a method for constructing AAV8-CRISPR/Cas9 system, including the following steps:

using restriction enzyme to cut an adeno-associated virus plasmid, obtaining a linear adeno-associated virus plasmid;

inserting the gRNA into the linear adeno-associated virus plasmid, obtaining the AAV8-CRISPR/Cas9 system.

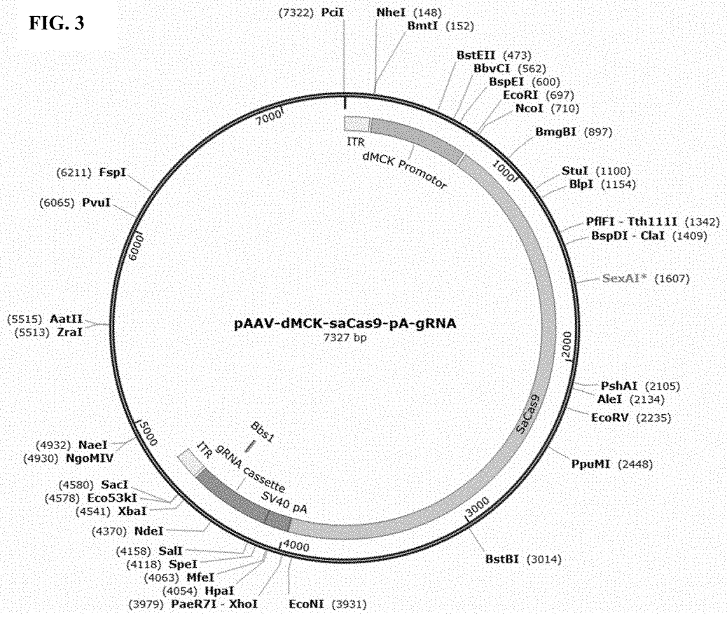

In an embodiment, the adeno-associated virus plasmid is selected as pAAV-dMCK-SACas9-PA-gRNA. The schematic structure of the adeno-associated virus plasmid can be found in FIG. 3. The pAAV-dMCK-SACas9-PA-gRNA is prepared by replacing LP1 promoter on plasmid with dMCK promoter based on pAAV-LP1-saCas9-pA-gRNA, and the specific dMCK promoter fragment is obtained from pAAV-dMCK-GFP by enzyme digestion with restriction enzyme NheI and EcorI, and the LP1 on pAAV-LP1-saCas9-pA-gRNA is excised by restriction enzyme NheI and EcorI, and the obtained dMCK promoter fragment is connected to the linearized vector, thus obtaining the pAAV-dMCK-SACas9-PA-gRNA. The pAAV-LP1-saCas9-pA-gRNA and pAAV-dMCK-GFP have been reported in the prior art (see also Prevention of Muscle Wasting by CRISPR/Cas9-mediated Disruption of Myostatin In Vivo; DOI: 10.1038/mt.2016.192).

In an embodiment, the restriction enzyme is BbsI. There is no special limit on the source of the enzyme involved, which means that any conventional route from which biochemical reagents known in the art are purchased of will do.

In an embodiment, the linear adeno-associated virus plasmid inserted with the gRNA is packaged into a recombinant AAV8 virus with the aid of AAV8 capsid plasmid and Helper plasmid. There is no special limit on the packaging method, any packaging method known in the art will do. The parts with muscle atrophy are injected with recombinant AAV8 virus to realize gene therapy of muscle atrophy.

The application provides use of the gRNA, the AAV8-CRISPER/Cas9 system or the AAV8-CRISPR/Cas9 system constructed by the above method in preparing drugs for treating muscle atrophy.

In embodiments, the muscle atrophy includes myogenic muscle atrophy, disused muscle atrophy or muscle atrophy induced by chronic disease heart failure.

In the application, the drug includes a reagent containing the recombinant AAV8 virus. The injection dose of the drug is 10¹¹VG per mouse.

The gRNA targeting miR-29b, AAV8-CRISPER/Cas9 system and use thereof provided in the application will be described clearly and completely in combination with the embodiments below, but they cannot be understood as a limit on the protection scope of the application.

### Example 1

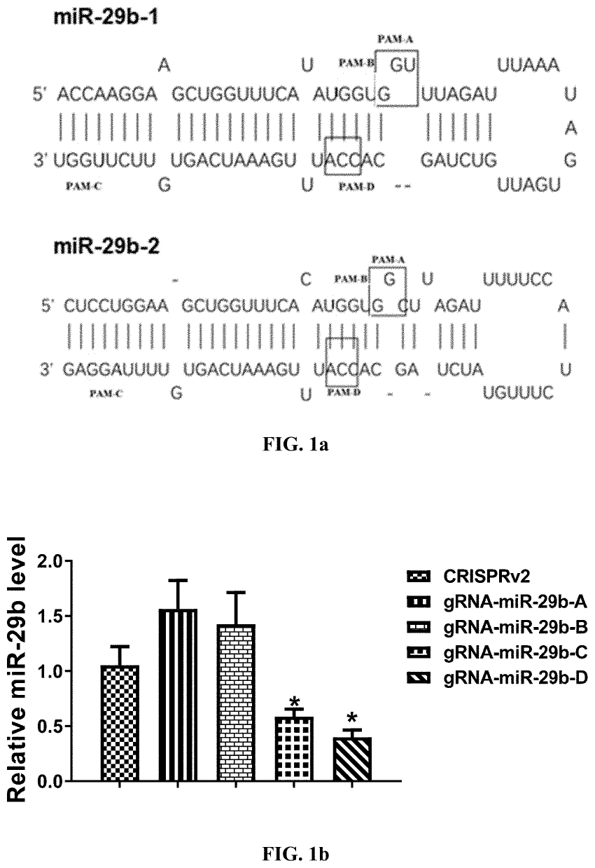

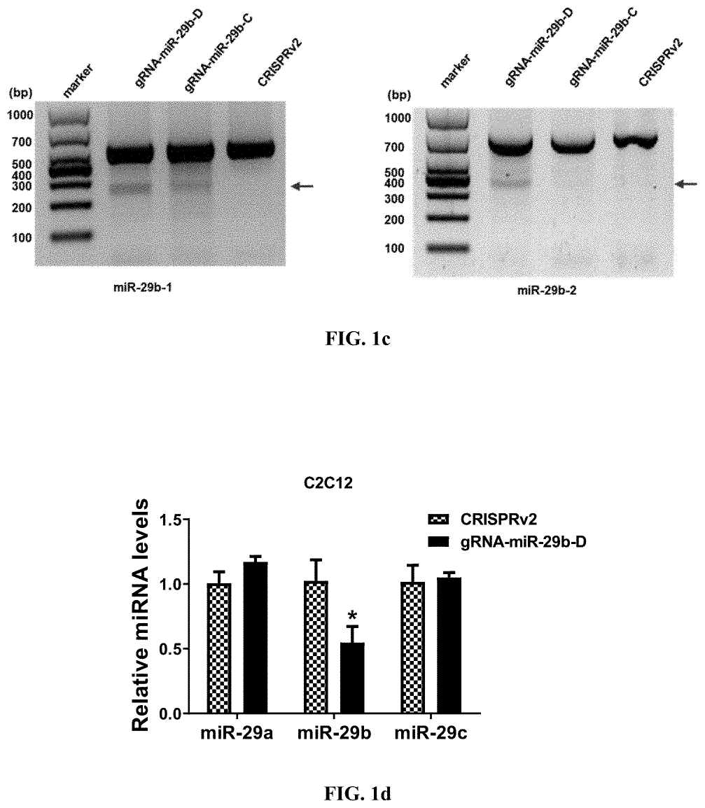

1. Design and synthesis of gRNA: gRNA was designed according to different editing sites of miR-29b (see in FIG. 1a), miR-29b-1 and miR-29b-2 indicated two gene sequences on chromosome 6 and chromosome 1, respectively, both of which may form mature miR-29b. PAM was called protospacer-adjacent motif, which was the recognition site of Cas9 enzyme. The PAM-A, PAM-B, PAM-C and PAM-D showed in the box were Cas9 enzyme recognition sites in the sequence.

The following four different gRNAs were designed: gRNA-miR-29b-A, gRNA-miR-29b-B, gRNA-miR-29b-C and gRNA-miR-29b-D, wherein the nucleotide sequences were respectively as follows: AGGAAGCTGGTTTCATATGG (SEQ ID No.4), TTCAGGAAGCTGGTTTCATA (SEQ ID No.5), CCATTTGAAATCAGTGTTTT (SEQ ID No.6), CCTAAAACACTGATTTCAAA (SEQ ID No.1).

2. C2C12 cell culture: DMEM complete medium containing 10% FBS and 1% P/S was used as the cell growth medium, and DMEM complete medium containing 2% horse serum and 1% P/S was used as the medium for inducing cell differentiation, and time for the differentiation was generally 4 days.

3. Effects of different gRNAs on the expression of miR-29b

The four gRNAs designed and synthesized above were inserted into Lenti-CRISPRv2 vector (purchased from addgene) to form the Lenti-CRISPR/Cas9-gRNA-miR-29b-A, Lenti-CRISPR/Cas9-gRNA-miR-29b-B, Lenti-CRISPR/Cas9-gRNA-miR-29b-C and Lenti-CRISPR/Cas9-gRNA-miR-29b-D, and then the four recombinant plasmids including Lenti-CRISPRv2 vector were transfected into C2C12 cells by lentivirus. The expression of miR-29b in C2C12 cells was detected by real-time fluorescence quantitative PCR, and the expression of miR-29b was calculated by 2-^{ΔΔCt} method with 5S as the internal reference. The specific method was as follows: firstly, the total RNA in cells was extracted by TRIzol Reagent, then 400 ng RNA was added with specific RT primers (purchased from Ruibo Company), and the reverse transcription experiment was proceeded by using reverse transcription kit of Bio-rad Company. The reverse transcription reaction system was as follows:

| | | |
|---|---|---|
| 400 ng RNA | 5.5 | μl |
| miR-29b RT primer (5 nM) | 1 | μl |
| 5s RT primer (5 nM) | 1 | μl |

The volume of the mixture was 7.5 μl, allowing the system at 70° C. for 10 min to fully open the stem-loop structure of RT primer, and annealed at 4° C. for 5 min, so that the miRNA was fully combined with specific RT primer.

Then, 2 μl of 5×iScript reaction mixture and 0.5 μl of iScript reverse transcriptase were added to said mixture in the previous step, followed by the following procedure:

| | | | |
|---|---|---|---|
| Priming | 25° C. | 5 | min |
| Reverse Transcription | 46° C. | 20 | min |
| RT inactivation | 95° C. | 1 | min |

The RT product was diluted 400-fold, and the expression of miR-29b was detected by qPCR method as follows.

The qPCR reaction system was prepared, the volume of which was 10 μl:

| | |
|---|---|
| SYBR Green | 5 μl |
| Primer mix F + R (5 nM) | 1 μl |
| cDNA (Diluted) | 4 μl |

Among them, miR-29b, the upstream and downstream primers of internal reference 5S were purchased from Ruibo Company.

The qPCR reaction procedure was as follows:

| | | | |
|---|---|---|---|
| 95° C. | 5 | min | Pre-degeneration |
| 95° C. | 10 | s | Circular reaction |
| 60° C. | 15 | s | (40 cycles) |
| 72° C. | 10 | s | |
| 95° C. | 15 | s | Dissociation curve |
| 60° C. | 60 | s | |
| 95° C. | 15 | s | |

The results are shown in FIG. 1b. As can be seen in FIG. 1b, compared with the control, the expression level of miR-29b in gRNA-miR-29b-A and gRNA-miR-29b-B treatment groups increases, while that in gRNA-miR-29b-C and gRNA-miR-29b-D treatment groups decreases significantly. Therefore, gRNA-miR-29b-C and gRNA-miR-29b-D are used for further experiments.

4. T7 enzyme digestion to verify the editing efficiency of gRNA-miR-29b-D and gRNA-miR-29b-C

Principle of gene editing: the gRNA is combined with Cas9, and the combination (gRNA-Cas9) targets to the DNA fragment that forms miR-29b. After the gRNA-Cas9 enters the nucleus and recognizes the PAM sequence and the target sequence, the DNA is cut off at the site three bases behind the combination, and a blunt-ended incision is produced. Broken DNA molecules mutate and lose their activity in the subsequent repair process of non-homologous end joining (NHEJ), thus realizing the silencing of the target gene. gRNA-miR-29b-D and gRNA-miR-29b-C here are different gRNAs that guide Cas9 to cut the target gene.

100 ng of mouse DNA (extracted from the mouse myoblast C2C12 by using tissue genomic DNA extraction kit) was used as template to amplify the targeted miR-29b-1 and miR-29b-2 genomic DNA fragments. 100 ng miR-29b-1 or miR-29b-2 gene fragment was respectively used as template, and the following primers were used for amplification: miR-29b-1 F 5′-GCTGCACCGTGAATGGTAA-3′ (SEQ ID No.7), R 5′-AGGTCTTCATCCGAGCATGG-3′ (SEQ ID No.8); miR-29b-2, F 5′-TGTACATATGTTGAATGGATTTGGT-3′(SEQ ID No.9), R5′-TGCTGCAACCAGGACTGAAT-3′(SEQ ID No.10).

The purified PCR products were denatured, and annealed in 20 μl NEB Buffer 2 under the following conditions: 95° C., 5 min; 95° C.˜75° C., 0.1° C.: 200 cycles; 75° C.˜15° C. 0.1° C.: 600 cycles; heat preservation at 4° C. 1U T7EN1 enzyme was added to the hybridized PCR product, and the reaction mixture was incubated at 37° C. for 1 hour. The product was separated with 2% agarose gel electrophoresis and stained with GelRED, as shown in FIG. 1c. The result shows that the editing efficiency of gRNA-miR-29b-D is obviously higher than that of gRNA-miR-29b-C.

5. Specificity test of gRNA-miR-29b-D to miR-29b

The Lenti-CRISPR/Cas9-GRNA-miR-29b-D virus was transfected into C2C12 cells by lentivirus, and the expression of miR-29a, miR-29b and miR-29c in miR-29 family was detected 72 hours after transfection, with the CRISPRv2 as the control. The specific method was as follows:

RNA was extracted from C2C12 cells transfected for 72 h, and cDNA was obtained by reverse transcription. After the concentration of the cDNA was quantitatively detected by nucleic acid, the cDNA was diluted to 200˜400 ng/μl, and then qPCR reaction was proceeded as follows.

The qPCR reaction system was:

| | | |
|---|---|---|
| SYBR Green | 5 | μl |
| Primer mix F+R (5 nM each) | 1 | μl |
| cDNA (diluted) | 4 | μl |

The qPCR reaction procedure was as follows:

Pre-denaturation at 95° C. for 5 min; denaturation at 95° C. for 10 s, anneal at 60° C. for 15 s, extension at 72° C. for 10 s, 40 cycles; 95° C. for 15 s, 60° C. for 60 s, 95° C. for 15 s, performing dissociation curve analysis. Three microRNA specific primers and specific reverse transcription primers were all purchased from Guangzhou Ruibo Biotechnology Co., Ltd, which did not provide specific primer sequences. As an internal reference of the qPCR reaction, 5S was also purchased from Guangzhou Ruibo Biotechnology Co., Ltd.

The result of qPCR is shown in FIG. 1d. It can be seen from FIG. 1d that gRNA-miR-29b-D only has targeted editing effect on miR-29b with no effect on miR-29a and miR-29c of the same family.

6. Verify whether gRNA-miR-29b-D has off-target effect

1) the Top10 positions where the gRNA-miR-29b-D may miss the target was detected whether the off-target occurs.

100 ng mouse gDNA was used as template to amplify DNA fragments. The primer used were shown in Table 1.

Table 1
Primer information for detecting Top10 positions where gRNA-miR-29b-D may
miss the target

| No. | Forward primer (5′-3′) | Reverse primer (5′-3′) | Amplification sites |
|---|---|---|---|
| 1 | GGGACTCGAGCACTCTATGTT | AGGGTCATTAGCAGGGTTCC | chr1: +196863288 |
| | (SEQ ID No. 13) | (SEQ ID No. 14) | |
| 2 | TCATCGGACCTTGACAGCTC | TGTGCCAGGCCAGAGAAAAA | chr9: −101867160 |
| | (SEQ ID No. 15) | (SEQ ID No. 16) | |
| 3 | AGCCGAGCTATCAATGGGC | TTCATCTGCATTCTGCGCTGT | chr2: −44912669 |
| | (SEQ ID No. 17) | (SEQ ID No. 18) | |
| 4 | CTGCATTGAGTGCCTTAGCG | GACCATTTGGAAACCGTGTGA | chr11: −26661876 |
| | (SEQ ID No. 19) | (SEQ ID No. 20) | |
| 5 | GCACTGGGGACATAGGTGAG | AGCCCACCTTGGCAATAGAC | chr5: −68275574 |
| | (SEQ ID No. 21) | (SEQ ID No. 22) | |
| 6 | CAGTGAGCTTCACAGTTTGCT | GAGTCATACAGTATTTAGGCTGC | chrX: +93743993 |
| | (SEQ ID No. 23) | T (SEQ ID No. 24) | |
| 7 | AAGGCTGAATGCCGTTCACT | GGCAAGAAGAACCTGGGACA | chr16: +15872481 |
| | (SEQ ID No. 25) | (SEQ ID No. 26) | |
| 8 | ATGCAGCAGATGCCAGACTT | CTCATGAGCACAGGAGCCAA | chr15: −66654630 |
| | (SEQ ID No. 27) | (SEQ ID No. 28) | |
| 9 | GACCACCACAATCGGCTGTA | GTTCTTGGCTCCCCTGACTC | chr11: −98951837 |
| | (SEQ ID No. 29) | (SEQ ID No. 30) | |
| 10 | TGGCTGCCAATACCTATGCT | AGCCATCCCCTCGACTCAAA | chr4: +40629962 |
| | (SEQ ID No. 31) | (SEQ ID No. 32) | |

The purified PCR product was denatured, and annealed in 20 μl NEB Buffer under the following conditions: 95° C., 5 min; 95° C.˜75° C., 0.1° C.: 200 cycles; 75° C.˜15° C., 0.1° C.: 600 cycles; heat preservation at 4° C. 1U T7EN1 enzyme was added to the hybridized PCR product, and the reaction mixture was incubated at 37° C. for 1 hour. The product was separated with 2% agarose gel electrophoresis and stained with GelRED, as shown in FIG. 1e.

The results are shown in FIG. 1e. As can be seen from FIG. 1e, all the 10 DNA fragments that may be off-target do not produce fragments cut by T7EN1 enzyme, which indicates that gRNA-miR-29b-D does not miss target.

2) miR-29a and miR-29c, members of the same family of miR-29b, were detected to confirm whether affected or not, for specific method, see also the method in step5 above, the result was shown in FIG. 1d.

### Example 2

Method for Constructing Recombinant Virus

Construction of recombinant plasmid: the plasmid pAAV-dMCK-saCas9-pA-gRNA was cut by BbsI restriction enzyme, and the above four designed gRNA sequences were respectively inserted into the plasmid, then the plasmid was transformed into competent cells, and four target plasmids containing gRNA were obtained after the sequencing of the selected monoclones.

Packaging of AAV8-CRISPR-Cas9-miR-29b-gRNA virus: 293T cells were inoculated in a 10 cm cell culture dish at a density of 4 million cells per culture dish. 24 hours later, 1 ml DMEM medium containing 10 μg AAV8, 10 μg pAAV-dMCK-saCas9-pA-gRNA, 10 μg Helper and 90 μg PEI MAX was added to each culture dish for transfection. After 12 hours of transfection, the medium was replaced by fresh DMEM complete medium, and the viruses in cells and cultures were collected after 48 hours. Collect virus in culture medium: 25 ml of 40% PEG-8000 solution was added to every 100 ml of culture medium, the mixture was stirred at 4° C. overnight, centrifuged (2800 g) at 15° C. for 15 min. 1 ml cell lysis buffer was added to the virus precipitate to resuspend. Collection of virus in cells: cell precipitation was resuspended in 5 ml cell lysis buffer, and cells were repeatedly undergone freeze-thaw in −80° C. refrigerator and 37° C. water bath for 3 times. The virus suspension in the culture medium was mixed with the freeze-thaw cell suspension, 1M MgCl₂ was added to the final concentration of 1 mM, Benzonase was added to the final concentration of 250 U/ml. Then the supernatant was collected after incubation at 37° C. for 45 minutes and centrifugation at 4° C. for 4 minutes at 4000 rpm. The virus was purified by gradient density centrifugation with iodixanol. See also FIG. 2a for the schematic diagram of the purified AAV8 virus carrying the target gene.

Detection method of virus titer: the virus vector plasmid pAAV-dMCK-saCas9-pA-gRNA was diluted to 1 ng/μl, and the concentration of the plasmid was calculated to be 1.36×10¹¹ VG/ml. The plasmids were diluted by 2-fold gradient for 13 times to obtain standards 1˜14, and the Standard DNA dilution by the 2-fold dilution was used to prepare standard curves.

5 μl purified virus AAV8-CRISPR-Cas9-miR-29b-gRNA prepared above was taken, and the virus gDNA was extracted by tissue genomic DNA extraction kit according to the instruction manual. The gDNA was finally eluted by 50 μl ddH₂O, and then the virus gDNA was diluted 100-fold, and then the virus titer was detected by qPCR method. The qPCR reaction system was as follows:

| | | |
|---|---|---|
| SYBR Green | 5 | μl |
| Upstream primer F and downstream primer R (10 μM) | 0.5 | μl |
| ddH₂O | 2.5 | μl |
| Standard DNA diluent or virus genome DNA | 2 | μl |
| Total volume | 10 | μl |

Sequences of upstream primer F and downstream primer R for qPCR reaction were as follows:

Upstream primer F:
(SEQ ID No. 11)
TACAACGCCCTGAATGACCT;
A downstream primer R:
(SEQ ID No. 12)
GTCCTCTTCGTTGACCGGA.

The qPCR reaction procedure was as follows: pre-denaturation at 95° C. for 10 min; denaturation at 95° C. for 15 s, anneal at 60° C. for 30 s, 40 cycles.

Because of the linear relationship between the number of cycles and logarithm of Standard DNA dilution concentration, the virus titer was obtained through linear fitting.

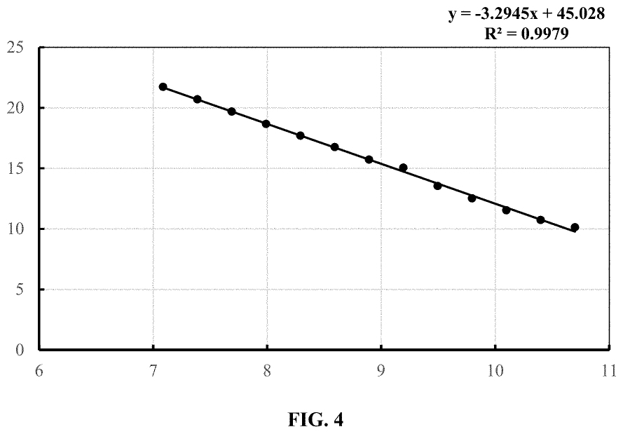

The standard curve was shown in FIG. 4. The Cq values of virus samples were 11.71, 11.74 and 11.76 (three duplicate samples), from which the virus titer was 2.36×10¹³ VG/ml.

The AAV8 virus after titer determination can be directly used in animal experiments or frozen at −80° C.

### Example 3

The experimental groups were divided into control group virus (excluding gRNA virus)+muscle atrophy model control group, control group virus+muscle atrophy model group, AAV8-CRISPR-Cas9-29b-D virus group+muscle atrophy model control group, and AAV8-CRISPR-Cas9-29b-D virus group+muscle atrophy model group.

The viruses (or viruses without gRNA) were directly injected into gastrocnemius muscle of mice at a dose of 1×10¹¹ VG per mouse, and muscle atrophy model was constructed three weeks later, wherein VG stood for vector genome. The muscle atrophy model induced by Ang II was constructed as follows: C57BL/6J mice were embedded with a slow-release pump (ALZET2001) containing Ang II (1.5 g/kg/min) in their backs, while PBS was filled in the slow-release pump embedded in the back of control mice. One week after the pump was embedded, the mice were killed and the gastrocnemius muscle was taken for test.

After the experiment, the grip strength of the hind limbs of the mice was detected by a grip strength meter, and then the mice were killed and dissected to obtain the gastrocnemius muscle, and the weight of the gastrocnemius muscle of the mice was weighed by an analytical balance. Afterwards frozen tissue was cut into slice, stained by WGA method, and the changes of muscle fiber cross-sectional area were counted. At the same time, gastrocnemius tissue samples were taken to extract the total RNA of gastrocnemius tissue, and the changes of miR-29b were detected by fluorescence quantitative PCR in step 2 of example 1.

Results:

1. The expression of mouse miR-29b

As shown in FIG. 2b, miR-29b increases significantly when AngII induces the gastrocnemius muscle in mice to atrophy. The AAV8-CRISPR-Cas9-29b-D virus can effectively reverse the rise of miR-29b.

2. Detection of mouse grip strength

As shown in FIG. 2c, AngII induces the gastrocnemius muscle in mice to atrophy, and the grip strength of mice decreases. The AAV8-CRISPR-Cas9-29b-D virus can effectively reverse muscle atrophy and restore partial grasping ability.

3. Muscle weight of gastrocnemius

As shown in FIG. 2d, AngII induces gastrocnemius muscle in mice to atrophy, and AAV8-CRISPR-Cas9-29b-D virus could effectively reverse muscle atrophy.

4. Diameter of myotube

As shown in FIG. 2e, AngII induces gastrocnemius muscle in mice to atrophy, and the myotube diameter of gastrocnemius muscle decreases significantly. The AAV8-CRISPR-Cas9-29b-D virus can effectively reverse muscle atrophy and restore the myotube diameter.

The above described are only preferred embodiments of the present application, It should be understood by those skilled in the art that, without departing from the principle of the present application, any variations and modifications fall into the scope of the present application.

1. An AAV8-CRISPR/Cas9 system targeting miR-29b, wherein the system comprises a gRNA targeting miR-29b.

2. The AAV8-CRISPR/Cas9 system according to claim 1, wherein the gRNA is expressed under dMCK promoter in the AAV8-CRISPR/Cas9 system.

3. A method for constructing the AAV8-CRISPR/Cas9 system according to claim 1, wherein the method comprises the following steps:
1) using restriction enzyme to cut an adeno-associated virus plasmid, obtaining a linear adeno-associated virus plasmid;
2) inserting the gRNA into the linear adeno-associated virus plasmid, obtaining the AAV8-CRISPR/Cas9 system.

4. The method according to claim 3, wherein the adeno-associated virus plasmid is pAAV-dMCK-SACas9-PA-gRNA.

5. The method according to claim 4, wherein the restriction enzyme is BbsI.

6. A method for treating muscle atrophy using the AAV8-CRISPER/Cas9 system according to claim 1.

7. The method according to claim 6, wherein the muscle atrophy comprises myogenic muscle atrophy, disused muscle atrophy, or muscle atrophy induced by chronic disease heart failure.

8. The system according to claim 1, wherein the nucleotide sequence of the gRNA is as set forth in SEQ ID No.1.

9. The system according to claim 8, wherein the nucleotide sequence of the miR-29b is as set forth in SEQ ID No.2.

10. The method according to claim 3, wherein the gRNA is expressed under a dMCK promoter in the AAV8-CRISPR/Cas9 system.

11. The method according to claim 10, wherein the adeno-associated virus plasmid is pAAV-dMCK-SACas9-PA-gRNA.

12. The method according to claim 6, wherein the gRNA is expressed under a dMCK promoter in the AAV8-CRISPR/Cas9 system.

13. The method according to claim 6, wherein the nucleotide sequence of the gRNA targeting miR-29b is as set forth in SEQ ID No.1.

14. The method according to claim 13, wherein the nucleotide sequence of the miR-29b is as set forth in SEQ ID No.2.

15. The method according to claim 6, wherein the AAV8-CRISPR/Cas9 system is constructed by the following steps:
1) using restriction enzyme to cut an adeno-associated virus plasmid, obtaining a linear adeno-associated virus plasmid; and
2) inserting the gRNA into the linear adeno-associated virus plasmid, obtaining the AAV8-CRISPR/Cas9 system.

16. The method according to claim 15, wherein the adeno-associated virus plasmid is pAAV-dMCK-SACas9-PA-gRNA.

17. The method according to claim 15, wherein the restriction enzyme is BbsI.
